(19) United States
(12) Patent Application Publication
(10) Pub. No.: US 2022/0073992 A1
(43) Pub. Date: Mar. 10, 2022

(54) DIAGNOSIS OF CHRONIC KIDNEY DISEASE (CKD) AND ITS SUBGROUPS

(71) Applicants: Hossein Abdul Tehrani, Tehran (IR); Alieh Gholaminejad, Isfahan (IR)

(72) Inventors: Hossein Abdul Tehrani, Tehran (IR); Alieh Gholaminejad, Isfahan (IR)

(73) Assignees: Hossein Abdul Tehrani Abdul Tehrani, Tehran (IR); Alieh Gholaminejad, Isfahan (IR); Tarbiat Modares University, Tehran (IR)

(21) Appl. No.: 17/532,157

(22) Filed: Nov. 22, 2021

Related U.S. Application Data
(60) Provisional application No. 63/117,469, filed on Nov. 24, 2020.

(51) Int. Cl. C12Q 1/6883

(52) U.S. Cl. CPC C12Q 1/6883; C12Q 2600/158; C12Q 2600/178

(57) ABSTRACT

A method for diagnosing chronic kidney disease (CKD), including measuring an expression level of each microRNA (miRNA) of first miRNAs in a biological sample of a subject and comparing the expression level of each miRNA of the first miRNAs with a respective reference level corresponding to each miRNA of the first miRNAs. The first miRNAs may include miR-30a-5p (SEQ ID NO: 1), miR-486-5p (SEQ ID NO: 2), miR-29c-3p (SEQ ID NO: 3), and miR-200c-3p (SEQ ID NO: 4). Determining the CKD is responsive to higher expression levels of the miR-30a-5p (SEQ ID NO: 1) and the miR-486-5p (SEQ ID NO: 2) and lower expression levels of miR-29c-3p (SEQ ID NO: 3) and miR-200c-3p (SEQ ID NO: 4) than the respective reference level.

### CROSS-REFERENCE TO RELATED APPLICATION

This application claims the benefit of priority from pending U.S. Provisional Patent Application Ser. No. 63/117,469, filed on Nov. 24, 2020, and entitled “MICRORNA BIOMARKER PANELS,” which is incorporated herein by reference in its entirety.

### TECHNICAL FIELD

The present disclosure generally relates to chronic kidney disease (CKD), particularly to a method and a kit for diagnosing CKD, and more particularly to microRNAs (miRNAs) for diagnosis of CKD and its subgroups.

### BACKGROUND

Chronic kidney disease (CKD) is one of the leading causes of death. Healthy kidneys filter waste and excess fluid from the blood and excrete it in the urine. In CKD, this filtration process is slowly disrupted because of damage to the kidneys over time, which causes increase in dangerous levels of fluid and waste in the body. Unfortunately, chronic kidney disease in the early stages may have no signs and symptoms, and this chronic illness may remain hidden until high levels of toxins are accumulated in the body. When kidney disease progresses, symptoms develop with time. It may eventually lead to end-stage renal failure, which is fatal without dialysis or a kidney transplant. As a result, early diagnosis is essential for successful treatment and control of the disease progress.

Although microalbuminuria and decrease in glomerular filtration rate (GFR) are two common indicators used for CKD diagnosis, both of these indicators have limited sensitivity and specificity and cannot be used for early-stage detection of the disease. For example, serum creatinine levels associated with the GFR do not exceed normal unless a high percentage of renal function is irreversibly impaired. It is also worth noting that serum creatinine levels are also affected by non-renal factors, such as age, sex, protein intake, and severe hepatic impairment, and do not have sufficient specificity for renal failure diagnosis. Currently, the only gold standard diagnostic test for assessing chronic kidney disease is kidney biopsy, which can also be used to determine the etiology and diagnose the disease's subtypes. However, this is an invasive test and comes with high levels of risk and cost. Therefore, early diagnosis of the CKD and its subgroups without the need for invasive biopsy remains a challenge.

One group of molecules that can play the biomarker role and have received much attention in recent years are microRNAs (miRNAs), which are dysregulated in various diseases. An ideal biomarker is that its presence or absence in biological fluids is strongly linked with the disease's onset and progression under investigation. Thus, according to the fact that the physiological and pathological regulation of intracellular miRNAs can alter the pattern of secreted miRNAs in body fluids, measuring miRNAs in biological fluids and their pathological changes allow the use of miRNAs as biomarkers of various diseases such as CKD.

Hence, there is a need for a specific and sensitive diagnostic panel of miRNAs that can detect CKD and differentiate between its subgroups in the early stage of the disease without any need for biopsy. Also, there is a need for a cost-effective and non-invasive method and a kit for diagnosing CKD and its subgroups using patients' body fluids using a diagnostic panel of miRNAs.

### SUMMARY

This summary is intended to provide an overview of the subject matter of the present disclosure and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary method for diagnosing chronic kidney disease (CKD). Exemplary method may include measuring an expression level of each microRNA (miRNA) of first miRNAs in a biological sample of a subject and determining a status of each miRNA by comparing the expression level of each miRNA of the first miRNAs with a respective reference level corresponding to each miRNA of the first miRNAs. In an exemplary embodiment, the first miRNAs may include miR-30a-5p (SEQ ID NO: 1), miR-486-5p (SEQ ID NO: 2), miR-29c-3p (SEQ ID NO: 3), and miR-200c-3p (SEQ ID NO: 4).

In an exemplary embodiment, determining the CKD may be responsive to higher expression levels of the miR-30a-5p (SEQ ID NO: 1) and the miR-486-5p (SEQ ID NO: 2) and lower expression levels of miR-29c-3p (SEQ ID NO: 3) and miR-200c-3p (SEQ ID NO: 4) than the respective reference level. In an exemplary embodiment, exemplary method may further include comparing an expression level of miR-216b-5p (SEQ ID NO: 5) with a respective reference level. In an exemplary embodiment, the miR-216b-5p (SEQ ID NO: 5) may have a lower expression level than the respective reference level in CKD.

In an exemplary embodiment, the higher expression level than the respective reference level may include an expression level higher than the respective reference level with a fold change of at least about 1.5 at a significance threshold of p<0.05. In an exemplary embodiment, the lower expression level than the respective reference level may include an expression level lower than the respective reference level with a fold change of up to about 0.55 at a significance threshold of p<0.05.

In an exemplary embodiment, exemplary method may further include determining a subgroup of the CKD. In an exemplary embodiment, determining the subgroup of the CKD may include measuring an expression level of each miRNA of second miRNAs in the biological sample of the subject, determining a status of each miRNA by comparing the expression level of each miRNA of the second miRNAs with a respective reference level corresponding to each miRNA of the second miRNAs, and determining the subgroup of CKD responsive to the status of each miRNA of the second miRNAs. In an exemplary embodiment, the subgroup of CKD may include one of diabetic nephropathy (DN), IgA nephropathy (IgAN), membranous nephropathy (MN), and focal segmental glomerulus nephropathy and minimal change disease (FSGS/MCD).

In an exemplary embodiment, the second miRNAs may include miR-126-3p (SEQ ID NO: 6), miR-26a-5p (SEQ ID NO: 7), miR-135b-5p (SEQ ID NO: 8) and let-7b-5p (SEQ ID NO: 9). In an exemplary embodiment, determining diabetic nephropathy (DN) may be responsive to a higher expression level of the miR-126-3p (SEQ ID NO: 6) and lower expression levels of the miR-26a-5p (SEQ ID NO: 7) and the miR-135b-5p (SEQ ID NO: 8) than the respective reference level.

In an exemplary embodiment, determining IgA nephropathy (IgAN) may be responsive to a higher expression level of the let-7b-5p (SEQ ID NO: 9) and a lower expression level of the miR-135b-5p (SEQ ID NO: 8) than the respective reference level. In an exemplary embodiment, determining membranous nephropathy (MN) may be responsive to higher expression levels of the let-7b-5p (SEQ ID NO: 9) and the miR-126-3p (SEQ ID NO: 6) than the respective reference level.

In an exemplary embodiment, determining minimal change disease and primary focal segmental glomerulosclerosis (MCD/FSGS) may be responsive to a higher expression level of the miR-135b-5p (SEQ ID NO: 8) and a lower expression level of the miR-126-3p (SEQ ID NO: 6) than the respective reference level.

In an exemplary embodiment, measuring the expression level of first miRNAs in the biological sample of the subject may include measuring the expression level of first miRNAs in the biological sample by conducting at least one of an amplification-based method, a hybridization-based method, and a sequencing method. In an exemplary embodiment, conducting at least one of the amplification-based method, the hybridization-based method, and the sequencing method may include conducting at least one of a real-time polymerase chain reaction (RT-PCR), an isothermal amplification method, a microarray assay, and next-generation sequencing (NGS). In an exemplary embodiment, measuring the expression level of first miRNAs in the biological sample of the subject may include measuring the expression level of first miRNAs in at least one of a urine sample, a blood sample, a serum sample, a plasma sample, and a kidney biopsy.

In another general aspect, the present disclosure describes an exemplary kit for diagnosing chronic kidney disease (CKD). Exemplary kit may include reagents for measuring an expression level of each microRNA (miRNA) of first miRNAs in a biological sample of a subject using a first set of oligonucleotides.

In an exemplary embodiment, the first set of oligonucleotides may be capable of specifically binding to the first miRNAs or their corresponding complementary DNA (cDNA). In an exemplary embodiment, the first miRNAs may include miR-30a-5p (SEQ ID NO: 1), miR-486-5p (SEQ ID NO: 2), miR-29c-3p (SEQ ID NO: 3), and miR-200c-3p (SEQ ID NO: 4). In an exemplary embodiment, the first miRNAs may further include miR-216b-5p (SEQ ID NO: 5).

In an exemplary embodiment, exemplary kit may further include reagents for measuring an expression level of each miRNA of second miRNAs using a second set of oligonucleotides. In an exemplary embodiment, the second set of oligonucleotides may be capable of specifically binding to second miRNAs or their corresponding cDNA. In an exemplary embodiment, the second miRNAs may include miR-126-3p (SEQ ID NO: 6), miR-26a-5p (SEQ ID NO: 7), miR-135b-5p (SEQ ID NO: 8) and let-7b-5p (SEQ ID NO: 9).

In an exemplary embodiment, the reagents for measuring the expression level of each miRNA of the first miRNAs and the second miRNAs may include reagents for measuring the expression level of each miRNA based on at least one of an amplification-based method, a hybridization-based method, and a sequencing method. In an exemplary embodiment, the reagents for measuring the expression level of each miRNA based on at least one of an amplification-based method, a hybridization-based method, and a sequencing method may include reagents for measuring the expression level of each miRNA based on at least one of a real-time polymerase chain reaction (RT-PCR), an isothermal amplification method, a microarray assay, and next-generation sequencing (NGS). In an exemplary embodiment, the first set of oligonucleotides and the second set of oligonucleotides may include at least one of immobilized oligonucleotides and detectably labeled oligonucleotides.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A illustrates an exemplary method for diagnosing chronic kidney disease (CKD), consistent with one or more exemplary embodiments of the present disclosure.

FIG. 1B illustrates an exemplary implementation of exemplary method for diagnosing CKD and its subgroups, consistent with one or more exemplary embodiments of the present disclosure.

FIG. 1C illustrates an exemplary implementation for determining a subgroup of the CKD in a subject, consistent with one or more exemplary embodiments of the present disclosure.

FIG. 2 illustrates an exemplary miRNA biomarker panel for diagnosing CKD and its subgroups, consistent with one or more exemplary embodiments of the present disclosure.

FIG. 3 illustrates an exemplary computer system in which an embodiment of the present disclosure, or portions thereof, may be implemented as computer-readable code, consistent with one or more exemplary embodiments of the present disclosure.

FIG. 4A illustrates an enrichment analysis of KEGG pathways in the form of a barograph, consistent with one or more exemplary embodiments of the present disclosure.

FIG. 4B illustrates an enrichment analysis of PANTHER pathways in a form of a barograph, consistent with one or more exemplary embodiments of the present disclosure.

FIG. 4C illustrates an enrichment analysis of GO processes in the form of a barograph, consistent with one or more exemplary embodiments of the present disclosure.

FIG. 5A illustrates expression changes of exemplary first miRNAs of an exemplary miRNA biomarker panel in healthy controls compared to CKD patients and MN, DN, IgAN, and FSGS/MCD subgroups, consistent with one or more exemplary embodiments of the present disclosure.

FIG. 5B illustrates expression changes of exemplary second miRNAs of an exemplary miRNA biomarker panel in healthy controls compared to all CKD patients and the MN, DN, IgAN, and FSGS/MCD subgroups, consistent with one or more exemplary embodiments of the present disclosure.

FIG. 6 illustrates receiver operating characteristic (ROC) curves of exemplary first miRNAs of an exemplary miRNA biomarker panel in CKD patients and healthy controls, consistent with one or more exemplary embodiments of the present disclosure.

FIG. 7A illustrates combined ROC curves of all miRNAs of first miRNAs including miR-30a-5p (SEQ ID NO: 1), miR-486-5p (SEQ ID NO: 2), miR-29c-3p (SEQ ID NO: 3), miR-200c-3p (SEQ ID NO: 4), and miR-216b-5p (SEQ ID NO: 5), consistent with one or more exemplary embodiments of the present disclosure.

FIG. 7B illustrates combined ROC curves of four miRNAs of first miRNAs including miR-30a-5p (SEQ ID NO: 1), miR-486-5p (SEQ ID NO: 2), miR-29c-3p (SEQ ID NO: 3), and miR-200c-3p (SEQ ID NO: 4), consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well-known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown but is to be accorded the widest possible scope consistent with the principles and features disclosed herein.

The present disclosure generally describes an exemplary microRNA (miRNA) biomarker panel, an exemplary kit, and an exemplary method for diagnosing chronic kidney disease (CKD) even in an early stage of the disease with an accuracy value of about 94%. In the present disclosure, exemplary miRNA biomarker panel, exemplary kit, and exemplary method may be used for diagnosing the CKD and its subgroups by examining biological samples of patients, particularly urine samples. Exemplary miRNA biomarker panel may be used for diagnosis of CKD subgroups, including diabetic nephropathy (DN), IgA nephropathy (IgAN), membranous nephropathy (MN), and focal segmental glomerulus nephropathy and minimal change disease (FSGS/MCD) with accuracy values of about 95%, 100%, 94%, and 84%, respectively.

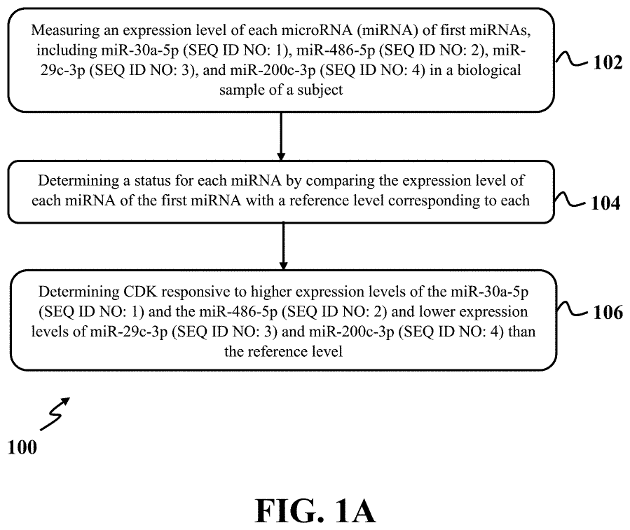

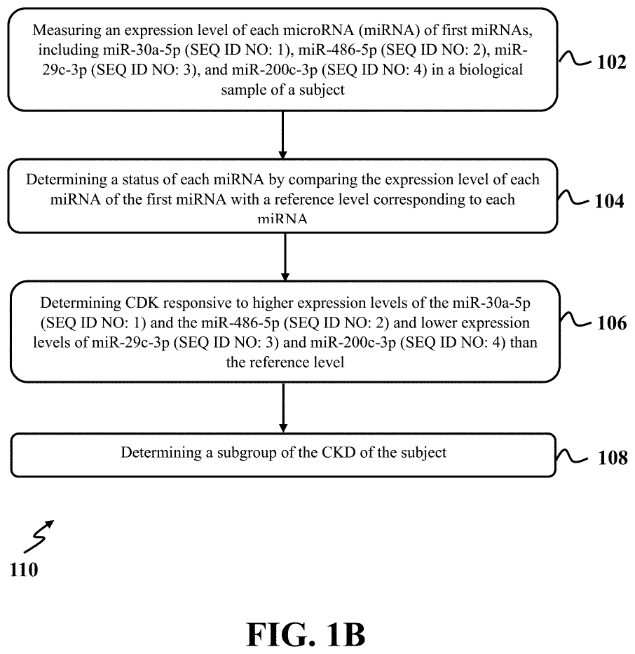

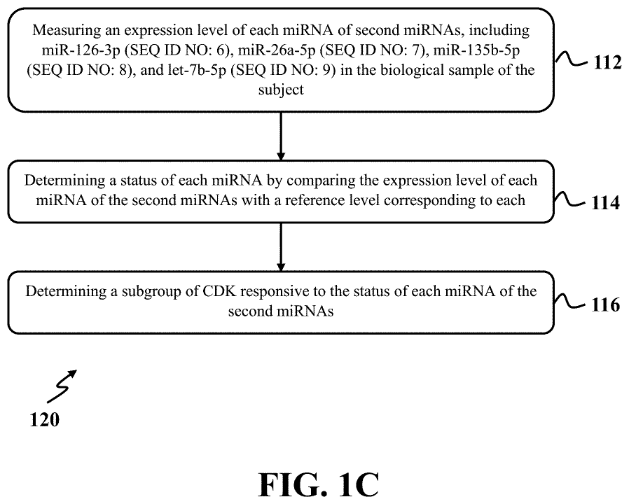

FIG. 1A illustrates an exemplary method 100 for diagnosing CKD, consistent with one or more exemplary embodiments of the present disclosure. Exemplary method 100 may include measuring an expression level of each miRNA of first miRNAs, including miR-30a-5p (SEQ ID NO: 1), miR-486-5p (SEQ ID NO: 2), miR-29c-3p (SEQ ID NO: 3), and miR-200c-3p (SEQ ID NO: 4) in a biological sample of a subject (step 102), determining a status of each miRNS by comparing the expression level of each miRNA of the first miRNAs with a respective reference level corresponding to each miRNA of the first miRNAs (step 104), and determining CKD responsive to higher expression levels of the miR-30a-5p (SEQ ID NO: 1) and the miR-486-5p (SEQ ID NO: 2) and lower expression levels of miR-29c-3p (SEQ ID NO: 3) and miR-200c-3p (SEQ ID NO: 4) than the respective reference level (step 106). In an exemplary embodiment, the first miRNAs may further include miR-216b-5p (SEQ ID NO: 5).

In further detail with respect to step 102, in an exemplary embodiment, measuring the expression level of each miRNA of the first miRNAs in the biological sample of the subject may include measuring the expression level of first miRNAs in the biological sample by conducting at least one of an amplification-based method, a hybridization-based method, and a sequencing method. In an exemplary embodiment, conducting the amplification-based method may include conducting at least one real-time polymerase chain reaction (RT-PCR) and an isothermal amplification method.

In the present disclosure, the expression level may be defined as “higher”, “lower”, or “unchanged” meaning that the subject expresses a certain miRNA at a level relative to a respective reference level. In the present disclosure, expression level of each miRNA may be obtained by calculating delta cycle threshold (ΔCt) through subtracting Ct of each miRNA from the Ct of one or more housekeeping genes as an internal control. In an exemplary embodiment, miR-448 (SEQ ID NO: 10) may be used as the internal control.

In an exemplary embodiment, conducting the RT-PCR may include extracting total RNA from the biological sample of the subject, synthesizing complementary DNA (cDNA) from the total RNA, and amplifying the cDNA corresponding to each miRNA using suitable primers, and detecting the expression level of each miRNA in the biological sample. In an exemplary embodiment, conducting the hybridization-based method may include conducting at least one of a microarray assay and a macroarray assay. In an exemplary embodiment, conducting the sequencing method may include conducting next-generation sequencing (NGS).

In an exemplary embodiment, measuring the expression level of first miRNAs in the biological sample of the subject may include measuring the expression level of first miRNAs in at least one of a urine sample, a blood sample, a serum sample, a plasma sample, and a kidney biopsy. In an exemplary embodiment, the biological sample may include a urine sample. In an exemplary embodiment, the urine sample may include at least one of urinary supernatant, urinary sediments, and urinary exosomes. In an exemplary embodiment, the blood sample may include at least one of a serum sample and a plasma sample.

In further detail with respect to step 104, in an exemplary embodiment, determining a status for each miRNA of the first miRNAs may include comparing the expression level of each miRNA of the first miRNAs with the respective reference level corresponding to each miRNA of the first miRNAs by calculating fold change of the expression level of each miRNA in comparison with the respective reference level corresponding to each miRNA of the first miRNAs through a computer-based computation system. In an exemplary embodiment, the respective reference level corresponding to each miRNA of the first miRNAs may include the expression level of that miRNA in healthy and normal samples. In an exemplary embodiment, comparing the expression level of each miRNA of the first miRNAs with the respective reference level corresponding to each miRNA of the first miRNAs may result in that each miRNA may have a higher expression level, lower expression level, or unchanged expression level relative to the respective reference level corresponding to each miRNA of the first miRNAs.

In an exemplary embodiment, comparing the expression level of each miRNA of the first miRNAs with the respective reference level corresponding to each miRNA of the first miRNAs may include calculating a fold change of the expression level of each miRNA of the first miRNAs relative to the respective reference level corresponding to each miRNA of the first miRNAs. In an exemplary embodiment, the fold change of the expression level of each miRNA may be calculated using delta-delta Ct method (2^{-ΔΔCt}), where ΔΔCt=ΔCt(sample)−ΔCt(reference).

In an exemplary embodiment, the status may include a higher expression level, a lower expression level, and an unchanged expression level. In an exemplary embodiment, the status may be the higher expression level if the fold change of the expression level of each miRNA of the first miRNAs to the respective reference level corresponding to each miRNA of the first miRNAs is at least about 1.5 at a significance threshold of p<0.05. In an exemplary embodiment, the higher expression level of each miRNA may indicate upregulation of that miRNA in the subject.

In an exemplary embodiment, the status may be the lower expression level if the fold change of the expression level of each miRNA of the first miRNAs to the respective reference level corresponding to each miRNA of the first miRNAs is up to about 0.55 at a significance threshold of p<0.05. In an exemplary embodiment, the lower expression level of each miRNA may indicate downregulation of that miRNA in the subject.

In further detail with respect to step 106, in an exemplary embodiment, determining CKD may be responsive to higher expression levels of the miR-30a-5p (SEQ ID NO: 1) and the miR-486-5p (SEQ ID NO: 2) and lower expression levels of miR-29c-3p (SEQ ID NO: 3) and miR-200c-3p (SEQ ID NO: 4) than the respective reference level corresponding to each miRNA of the first miRNAs. In an exemplary embodiment, the miR-216b-5p (SEQ ID NO: 5) may have a lower expression compared to the respective reference level corresponding to the miR-216b-5p (SEQ ID NO: 5) in the CKD.

In an exemplary embodiment, if the subject is diagnosed with the CKD based on exemplary method 100, a CKD subgroup of the subject may be determined based on status of second miRNAs. In an exemplary implementation, exemplary method 100 may further include determining a subgroup of the CKD of the subject. FIG. 1B illustrates an exemplary implementation of exemplary method 110 for diagnosing CKD and its subgroups, consistent with one or more exemplary embodiments of the present disclosure. Referring to FIG. 1B, exemplary method 110 may contain steps 102, 104, and 106 of method 100, in addition to determining a subgroup of the CKD of the subject (step 108).

In further detail with respect to step 108, in an exemplary embodiment, determining the subgroup of the CKD of the subject may include classifying the CKD of the subject into one of diabetic nephropathy (DN), IgA nephropathy (IgAN), membranous nephropathy (MN), and focal segmental glomerulus nephropathy and minimal change disease (FSGS/MCD). FIG. 1C illustrates an exemplary method 120 for determining the subgroup of the CKD in the subject, consistent with one or more exemplary embodiments of the present disclosure. Referring to FIG. 1C, exemplary method 120 may provide details of step 108 of method 110, where the exemplary process may comprise of measuring an expression level of each miRNA of second miRNAs, including miR-126-3p (SEQ ID NO: 6), miR-26a-5p (SEQ ID NO: 7), miR-135b-5p (SEQ ID NO: 8), and let-7b-5p (SEQ ID NO: 9) in the biological sample of the subject (step 112), determining a status of each miRNA by comparing the expression level of each miRNA of the second miRNAs with a respective reference level corresponding to each miRNA of the second miRNAs (step 114), and determining the subgroup of CKD responsive to the status of the second miRNAs (step 116).

In further detail with respect to step 112, in an exemplary embodiment, measuring the expression level of each miRNA of the second miRNAs in the biological sample of the subject may be conducted similar to step 102 for measuring the expression level of each miRNA of the first miRNAs in the biological sample of the subject.

In further detail with respect to step 114, in an exemplary embodiment, determining a status of each miRNA by comparing the expression level of each miRNA of the second miRNAs with the respective reference level corresponding to each miRNA of the second miRNAs may include calculating a fold change of the expression level of each miRNA of the second miRNAs relative to the respective reference level corresponding to each miRNA of the second miRNAs. In an exemplary embodiment, calculating fold change of the expression level of each miRNA in comparison with the respective reference level corresponding to each miRNA of the second miRNAs may include calculating fold change of the expression level of each miRNA through a computer-based computation system. In an exemplary embodiment, the fold change of the expression level of each miRNA may be calculated using delta-delta Ct method (2^{-ΔΔCt}), where ΔΔCt=ΔCt(sample)−ΔCt(reference).

In an exemplary embodiment, the respective reference level corresponding to each miRNA of the second miRNAs may include the expression level of that miRNA in healthy and normal samples. In an exemplary embodiment, comparing the expression level of each miRNA of the second miRNAs with the respective reference level corresponding to each miRNA of the second miRNAs may be conducted similar to step 104 for comparing the expression level of each miRNA of the first miRNAs with the respective reference level corresponding to each miRNA of the second miRNAs. In an exemplary embodiment, status of the second miRNAs may be used for differentiating between the subgroups of the CKD in the subject as described in step 116.

In an exemplary embodiment, comparing the expression level of each miRNA of the second miRNAs with the respective reference level corresponding to each miRNA of the second miRNAs may result in that each miRNA may have a higher expression level, lower expression level, or unchanged expression level relative to the respective reference level corresponding to each miRNA of the first miRNAs.

In an exemplary embodiment, the status may include a higher expression level, a lower expression level, and an unchanged expression level. In an exemplary embodiment, the status may be the higher expression level if the fold change of the expression level of each miRNA of the second miRNAs to the respective reference level corresponding to each miRNA of the second miRNAs is at least about 1.5 at a significance threshold of p<0.05. In an exemplary embodiment, the higher expression level of each miRNA may indicate upregulation of that miRNA in the subject.

In an exemplary embodiment, the status may be the lower expression level if the fold change of the expression level of each miRNA of the second miRNAs to the respective reference level corresponding to each miRNA of the second miRNAs is up to about 0.55 at a significance threshold of p<0.05. In an exemplary embodiment, the lower expression level of each miRNA may indicate downregulation of that miRNA in the subject.

In further detail with respect to step 116, determining the subgroup of CKD may be responsive to the status of the second miRNAs. Accordingly, in an exemplary embodiment, determining diabetic nephropathy (DN) may be responsive to a higher expression level of the miR-126-3p (SEQ ID NO: 6) and lower expression levels of the miR-26a-5p (SEQ ID NO: 7) and the miR-135b-5p (SEQ ID NO: 8) than the respective reference level. In an exemplary embodiment, determining IgA nephropathy (IgAN) may be responsive to a higher expression level of the let-7b-5p (SEQ ID NO: 9) and a lower expression level of the miR-135b-5p (SEQ ID NO: 8) than the respective reference level.

In an exemplary embodiment, determining membranous nephropathy (MN) may be responsive to higher expression levels of the let-7b-5p (SEQ ID NO: 9) and the miR-126-3p (SEQ ID NO: 6) than the respective reference level. In an exemplary embodiment, determining minimal change disease and primary focal segmental glomerulosclerosis (MCD/FSGS) may be responsive to a higher expression level of the miR-135b-5p (SEQ ID NO: 8) and a lower expression level of the miR-126-3p (SEQ ID NO: 6) than the respective reference level.

In an exemplary embodiment, exemplary miRNA biomarker panel may include a set of dysregulated miRNAs that provide the ability to diagnose CKD and its subgroups from healthy individuals. In an exemplary embodiment, exemplary miRNA biomarker panel may be used to diagnose CKD and its subgroups by implementing an exemplary method similar to method 100 of FIG. 1A and method 110 of FIG. 1B.

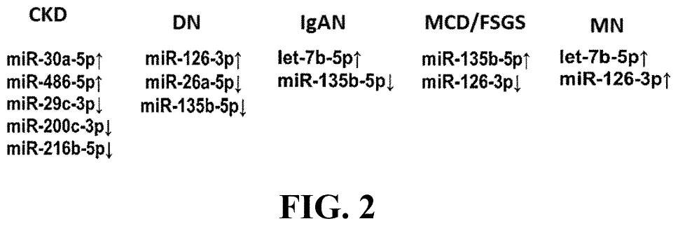

FIG. 2 illustrates an exemplary miRNA biomarker panel for diagnosing CKD and its subgroups, consistent with one or more exemplary embodiments of the present disclosure. Upward and downward arrows may be used to represent the status of each miRNA including the higher expression level and the lower expression level, respectively. Referring to FIG. 2, the miRNA biomarker panel may confirm the CKD if the biological sample of the subject has miR-30a-5p↑, miR-486-5p↑, miR-200c-3p↓, and miR-216b-5p↓. In an exemplary embodiment, exemplary miRNA biomarker panel may be used to confirm if an individual has CKD with a diagnostic accuracy of about 94%.

In an exemplary embodiment, the miRNA biomarker panel may confirm the DN if the biological sample of the subject has miR-126-3p↑, miR-26a-5p↓, and miR-135b-5p↓. In an exemplary embodiment, exemplary miRNA biomarker panel may be used to confirm if an individual has DN with a diagnostic accuracy of about 95%. In an exemplary embodiment, the miRNA biomarker panel may confirm the IgAN if the biological sample of the subject has let-7b-5p↑ and miR-135b-5p↓. In an exemplary embodiment, exemplary miRNA biomarker panel may be used to confirm if an individual has IgAN with a diagnostic accuracy of about 100%.

In an exemplary embodiment, the miRNA biomarker panel may confirm the MN if the biological sample of the subject has let-7b-5p↑, miR-126-3p↑. In an exemplary embodiment, exemplary miRNA biomarker panel may be used to confirm if an individual has MN with a diagnostic accuracy of about 94%. In an exemplary embodiment, the miRNA biomarker panel may confirm the FSGS/MCD if the biological sample of the subject has miR-135b-5p↑ and miR-126-3p↓. In an exemplary embodiment, exemplary miRNA biomarker panel may be used to confirm if an individual has FSGS/MCD with a diagnostic accuracy of about 84%.

In an exemplary implementation, exemplary methods 100, 110, and 120 may be carried out utilizing an exemplary kit for diagnosing CKD. In an exemplary embodiment, exemplary kit may be used for determining miRNA expression profiles of exemplary miRNA biomarker panel using biological samples of the subject. Exemplary kit may include reagents for measuring an expression level of each miRNA of first miRNAs in a biological sample using a first set of oligonucleotides. In an exemplary embodiment, exemplary kit may further include reagents for extracting or enriching the first and second miRNAs of the biological sample.

In an exemplary embodiment, the first set of oligonucleotides may be capable of specifically binding to the first miRNAs or their corresponding complementary DNA (cDNA). In an exemplary embodiment, first miRNAs may include miR-30a-5p (SEQ ID NO: 1), miR-486-5p (SEQ ID NO: 2), miR-29c-3p (SEQ ID NO: 3), and miR-200c-3p (SEQ ID NO: 4). In an exemplary embodiment, the first miRNAs may further include miR-216b-5p (SEQ ID NO: 5).

In an exemplary embodiment, an exemplary kit may further include exemplary reagents for measuring an expression level of each miRNA of second miRNAs using a second set of oligonucleotides. In an exemplary embodiment, the second set of oligonucleotides may be capable of specifically binding to second miRNAs or their corresponding cDNA. In an exemplary embodiment, the second miRNAs may include miR-126-3p (SEQ ID NO: 6), miR-26a-5p (SEQ ID NO: 7), miR-135b-5p (SEQ ID NO: 8) and let-7b-5p (SEQ ID NO: 9).

In an exemplary embodiment, the reagents for measuring the expression level of each miRNA of the first miRNAs and the second miRNAs may include reagents for measuring the expression level of each miRNA based on at least one of an amplification-based method, a hybridization-based method, and a sequencing method. In an exemplary embodiment, the reagents for measuring the expression level of each miRNA based on the amplification-based method may include reagents for measuring the expression level of each miRNA based on at least one of an RT-PCR and an isothermal amplification method.

In an exemplary embodiment, the reagents for measuring the expression level of each miRNA based on the hybridization-based method may include reagents for measuring the expression level of each miRNA based on at least one of a microarray assay and a macroarray assay. In an exemplary embodiment, the reagents for measuring the expression level of each miRNA based on the sequencing method may include reagents for measuring the expression level of each miRNA based on NGS. In an exemplary embodiment, the first sets of oligonucleotides and the second sets of oligonucleotides may include at least one of a polymerase chain reaction (PCR) primer and a probe.

In an exemplary embodiment, the first set of oligonucleotides and the second set of oligonucleotides may include at least one of immobilized oligonucleotides and detectably labeled oligonucleotides. In an exemplary embodiment, the detectably labeled oligonucleotides may include oligonucleotides detectably labeled with at least one of an isotope and a fluorescent agent. In an exemplary embodiment, each of the detectably labeled oligonucleotides may be configured to generate a measurable signal in the presence of each miRNA for measuring the expression level of each miRNA. In an exemplary embodiment, the immobilized oligonucleotides may include oligonucleotides immobilized on a substrate. In an exemplary embodiment, the substrate may include at least one of a membrane, a chip, a disk, a strip, a filter, a microsphere, a slide, a multi-well plate, a bead, and an optical fiber.

In an exemplary embodiment, exemplary methods 100, 110, and 120, particularly steps 104, 106, 108, 114, and 116 may be implemented using an exemplary computer system. In an exemplary embodiment, exemplary computer system may include means for receiving data representing an expression level of each miRNA of the first miRNAs and the second miRNAs. In an exemplary embodiment, exemplary computer system may include means for receiving data representing the respective reference level corresponding to each miRNA of the first miRNAs and the second miRNAs.

In an exemplary embodiment, exemplary computer system may include means for comparing the expression level of each miRNA with the respective reference level corresponding to each miRNA of the first miRNAs and the second miRNAs. In an exemplary embodiment, exemplary computer system may also include means for determining the CKD and its subgroups in the subject. In an exemplary embodiment, the respective reference level corresponding to each miRNA of the first miRNAs and the second miRNAs may include at least one digital or numerical information. In an exemplary embodiment, the respective reference level corresponding to each miRNA of the first miRNAs and the second miRNAs may be provided in any readable or electronically readable form, including, but not limited to printed form, electronically stored form on a computer readable medium, such as CD, smart card, or provided in a downloadable form in a computer network such as the internet.

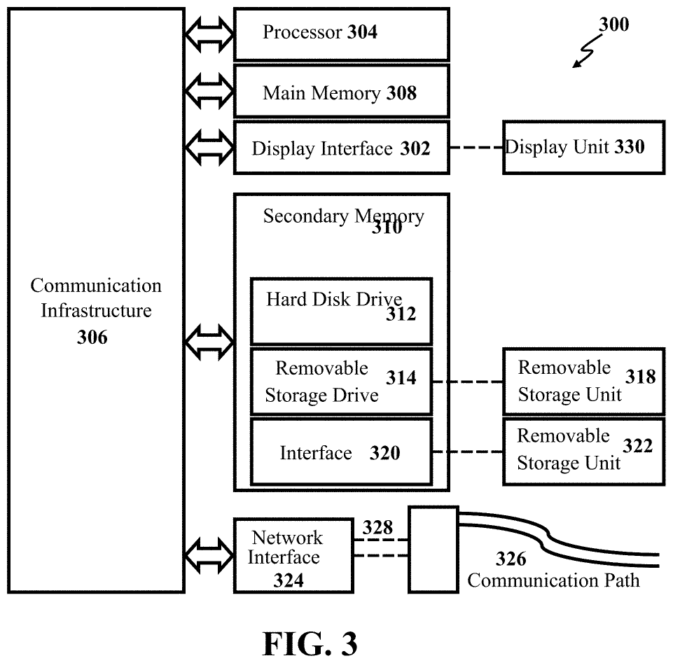

FIG. 3 illustrates an exemplary computer system 300 in which an embodiment of the present disclosure, or portions thereof, may be implemented as computer-readable code, consistent with one or more exemplary embodiments of the present disclosure. For example, steps 104, 106, 108, 114, and 116, of flowcharts presented in methods 100, 110, and 120 may be implemented in computer unit 300 using hardware, software, firmware, tangible computer-readable media having instructions stored thereon, or a combination thereof and may be implemented in one or more.

If programmable logic is used, such logic may execute on a commercially available processing platform or a particular purpose device. One ordinary skill in the art may appreciate that an embodiment of the disclosed subject matter can be practiced with various processor configurations, including multi-core multiprocessor systems, minicomputers, mainframe computers, computers linked or clustered with distributed functions, as well as pervasive or miniature computers that may be embedded into virtually any device.

For instance, a computing device with at least one processor device and a memory may implement the above-described embodiments. A processor device may be a single processor, a plurality of processors, or combinations thereof. Processor devices may have one or more processor “cores.”

An embodiment of the invention is described in terms of this example computer unit 300. After reading this description, it may become apparent to a person skilled in the relevant art how to implement the invention using other processors and/or computer architectures. Although operations may be described as a sequential process, some of the operations may be performed in parallel, concurrently, and/or in a distributed environment, and with program code stored locally or remotely for access by single or multiprocessor machines. In addition, in some embodiments, the order of operations may be rearranged without departing from the spirit of the disclosed subject matter.

Processor device 304 may be a special purpose or a general-purpose processor device. As may be appreciated by persons skilled in the relevant art, processor device 304 may also be a single processor in a multi-core/multiprocessor system, such system operating alone or in a cluster of computing devices operating in a cluster or server farm. Processor device 304 may be connected to a communication infrastructure 306, for example, a bus, message queue, network, or multi-core message-passing scheme.

In an exemplary embodiment, computer unit 300 may include a display interface 302, for example, a video connector, to transfer data to a display unit 330, for example, a monitor. Computer unit 300 may also include a main memory 308, for example, random access memory (RAM), and may also include a secondary memory 310. Secondary memory 310 may include, for example, a hard disk drive 312 and a removable storage drive 314. Removable storage drive 314 may include a floppy disk drive, a magnetic tape drive, an optical disk drive, a flash memory, or the like. Removable storage drive 314 may read from and/or write to a removable storage unit 318 in a well-known manner. Removable storage unit 318 may include a floppy disk, a magnetic tape, an optical disk, etc., which may be read by and written to by removable storage drive 314. As will be appreciated by persons skilled in the relevant art, removable storage unit 318 may include a computer-usable storage medium having stored therein computer software and/or data.

In alternative implementations, secondary memory 310 may include other similar means for allowing computer programs or other instructions to be loaded into computer unit 300. Such means may include, for example, a removable storage unit 322 and an interface 320. Examples of such means may include a program cartridge and cartridge interface (such as that found in video game devices), a removable memory chip (such as an EPROM, or PROM) and associated socket, and other removable storage units 322 and interfaces 320, which allow software and data to be transferred from removable storage unit 322 to computer unit 300.

Computer unit 300 may also include a communications interface 324. Communications interface 324 allows software and data to be transferred between computer unit 300 and external devices. Communications interface 324 may include a modem, a network interface (such as an Ethernet card), a communications port, a PCMCIA slot, card, or the like. Software and data transferred via communications interface 324 may be in the form of signals, which may be electronic, electromagnetic, optical, or other signals capable of being received by communications interface 324. These signals may be provided to communications interface 324 via a communications path 326. Communications path 326 carries signals and may be implemented using wire or cable, fiber optics, a phone line, a cellular phone link, an RF link, or other communications channels.

In this document, the terms “computer program medium” and “computer usable medium” are used to generally refer to media such as removable storage unit 318, removable storage unit 322, and a hard disk installed in hard disk drive 312. Computer program medium and computer usable medium may also refer to memories, such as main memory 308 and secondary memory 310, which may be memory semiconductors (e.g., DRAMs, etc.).

Computer programs (also called computer control logic) are stored in main memory 308 and/or secondary memory 310. Computer programs may also be received via communications interface 324. Such computer programs, when executed, enable computer unit 300 to implement different embodiments of the present disclosure as discussed herein. In particular, the computer programs, when executed, enable processor device 304 to implement the processes of the present disclosure, such as the operations in method 100 illustrated by flowchart 100 of FIG. 1A discussed above. Accordingly, such computer programs represent controllers of computer unit 300. Where an exemplary embodiment of method 100 is implemented using software, the software may be stored in a computer program product and loaded into computer unit 300 using removable storage drive 314, interface 320, and hard disk drive 312, or communications interface 324.

Embodiments of the present disclosure also may be directed to computer program products, including software stored on any computer useable medium. Such software, when executed in one or more data processing devices, causes a data processing device to operate as described herein. An embodiment of the present disclosure may employ any computer useable or readable medium. Examples of computer useable mediums include, but are not limited to, primary storage devices (e.g., any type of random-access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

### EXAMPLES

### Example 1: Bioinformatic Analysis of Exemplary MIRNA Biomarker Panel

In this example, exemplary miRNA biomarker panel was assessed using bioinformatic analysis to validate that miRNAs of the exemplary miRNA biomarker panel are present in the effective pathways of the CKD. In order to perform functional enrichment analysis of the miRNAs, a web tool was used to analyze the enrichment in the gene ontology (GO) processes and the Kyoto encyclopedia of genes and genomes (KEGG) and protein analysis through evolutionary relationships (PANTHER) pathways.

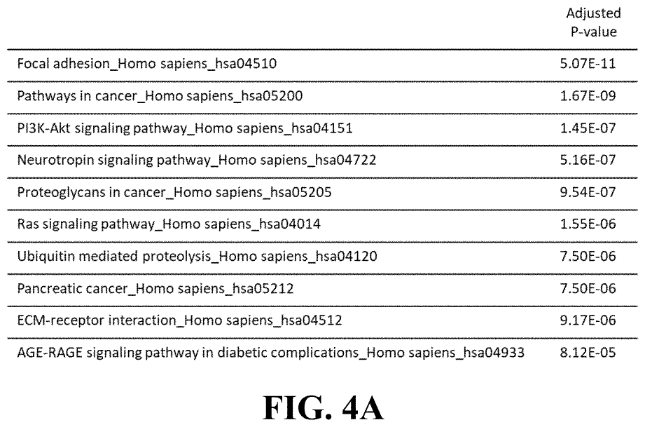

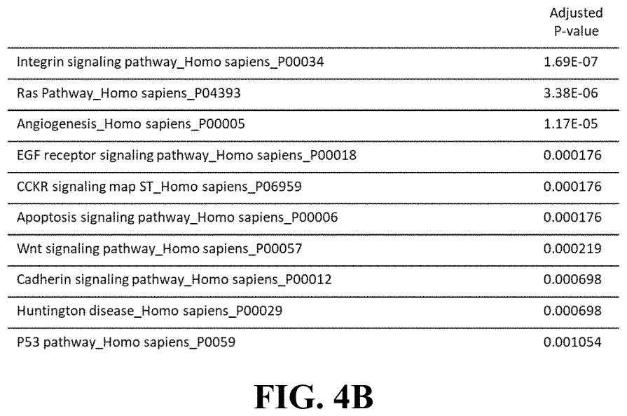

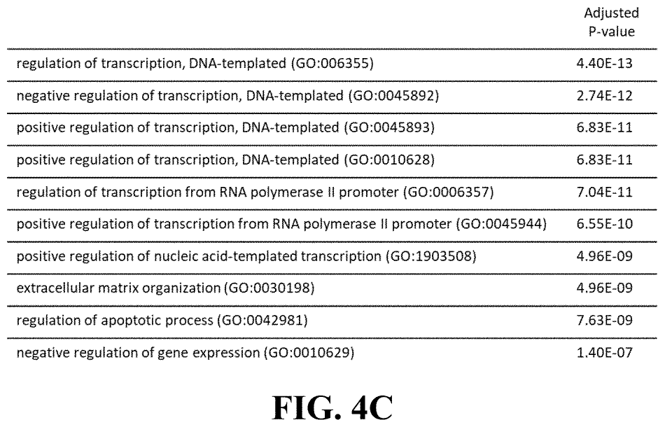

As input data, consensus target genes were used for each miRNA, and each pathway's adjusted p-values were obtained. FIG. 4A illustrates an enrichment analysis of KEGG pathways in the form of a barograph, consistent with one or more exemplary embodiments of the present disclosure. FIG. 4B illustrates an enrichment analysis of PANTHER pathways in a form of a barograph, consistent with one or more exemplary embodiments of the present disclosure. FIG. 4C illustrates an enrichment analysis of GO processes in the form of a barograph, consistent with one or more exemplary embodiments of the present disclosure.

Referring to FIGS. 4A-4C, the results showed that the enriched KEGG and PANTHER pathways are often associated with: cellular signaling pathways (PI3K-Akt signaling pathway, neurotrophin signaling pathway, Ras signaling pathway, AGE-RAGE signaling pathways in diabetic pathways, E integrin signaling pathway, EG integrating signaling pathway, CCKR signaling, Wnt signaling pathway, p53 pathway) along with cellular communication pathways (focal adhesion, ECM receptor interactions, cadherin signaling pathways, and proteoglycan pathways) and apoptotic process (10 pathways with the lowest adjusted P-values). Most GO-enriched processes regulated by the miRNAs include transcription regulation (positive and negative), regulation of gene expression, extracellular matrix organization, and the apoptotic process (10 processes with the lowest adjusted P-values).

Thus, enrichment analysis shows that these miRNAs can target a wide range of substrate genes. It is also essential to highlight the targets that focus on the cellular process associated with renal fibrosis, including hypertrophy and proliferation of mesenteric cells, EMT or endothelial to mesenchymal transfer, podocyte apoptosis, and isolation from the glomerular basement membrane. Analysis of the enriched KEGG and Panther pathways for miRNAs of exemplary miRNA biomarker panel showed that these miRNAs' target genes form highly rich networks with diverse pathways and molecule-molecule interaction, such as cellular signal and cellular motility and communication, at the fibrous cellular process. As a result, miRNAs of exemplary miRNA biomarker panel may be considered as potential biomarkers or therapeutic candidates for clinical use in CKD.

### Example 2: Experimental Validation of Exemplary Mirna Biomarker Panel

In this example, the exemplary miRNA biomarker panel was experimentally validated using real-time PCR on urine samples of healthy individuals and CKD patients. Amplification of microRNA with real-time PCR method is one of the most widely used techniques in miRNA research. However, the short length of miRNAs has made it difficult to detect and amplify them by conventional real-time PCR methods. Therefore, a special Bon-miR QPCR kit was used first to perform polyadenylation reaction and then reverse transcription. The polyadenylated RNA was converted to the corresponding cDNA, and finally, real-time PCR was done for the miRNAs.

The sampling process was performed for six months. Urine samples were taken from hospitalized patients from between 8 a.m. to noon on the day they underwent kidney biopsy at a fasting condition. The results of routine renal tests of each patient were extracted and recorded. After six months, four groups were selected from all groups of nephropathies with urine samples available, including 19 MN, 19 FSGS/MCD, 8 IgAN, and 4 DN patients. Meanwhile, because patients with diabetic nephropathy are less likely to undergo biopsy, DN patients in stages 1 and 2 nephropathies (GFR>60) were selected for urine collection, making the number of DN patient samples 14.

Besides, control urine samples were collected from a large number (about 50) of healthy individuals, and urine samples with blood or protein were excluded. The control samples were matched with the patient samples in terms of sampling time per day (between 8 a.m. in the morning to noon), age, and sex. All samples were taken in sterile urine containers, and the following preparation steps were performed within 1 to 2 hours. The samples were first centrifuged at 3000 g for 20 minutes to ensure that the urine samples do not have cells. The supernatant was transferred to 3 microtubes with volumes of 0.5 ml, 0.5 ml, and 1.5 ml. Falcon 15 was used to freeze and store 15 ml of urine at a temperature of about −80° C. Finally, 60 patient samples and 30 healthy control samples were collected, and all 90 samples entered the RNA extraction stage.

While biological fluids, especially urine, contain small amounts of miRNA and usually lack DNA and RNA molecules, extraction of this small amount of RNA requires more preparation than extraction from other tissues. According to the manufacturer's protocol, extraction of total RNA containing miRNAs was performed using the miTotal RNA Extraction Miniprep kit. While miRNAs are removed in conventional RNA extraction methods, either the buffers' pH may be adjusted, or suitable columns may be used to maintain miRNAs during the extraction.

During real-time PCR, a reverse primer is universal and binds to the sequence added to the miRNAs during cDNA synthesis. The forward primer determines the specificity of the real-time PCR reaction. Therefore, primers were designed and synthesized for miRNAs of exemplary miRNA biomarker panel. The mean expression of two internal controls, 5s rRNA and miR-448, which showed a stable expression in urine, was used to normalize cDNA changes in different samples and increase reliability.

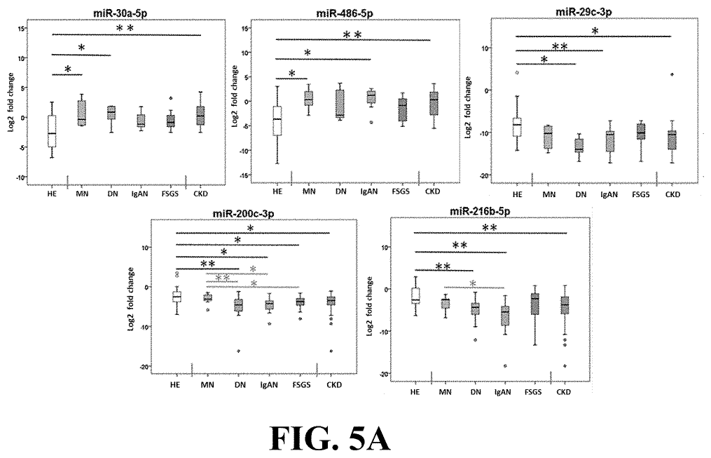

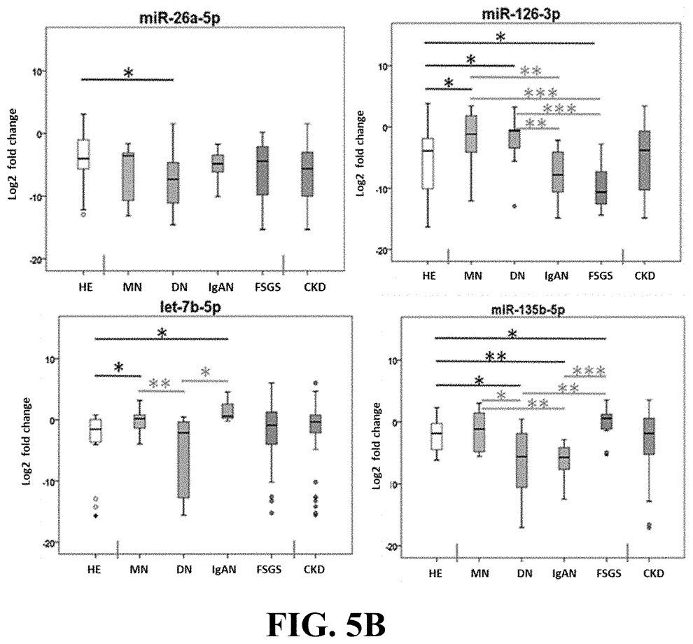

After the polyadenylation reaction and cDNA synthesis, the real-time PCR was done using the universal reverse primer and the miRNA-specific primers. The real-time PCR efficiency was evaluated by drawing a standard curve for each gene and two internal controls. Expression levels of miRNAs were obtained and analyzed by Mann-Whitney statistical test, and fold change (FC) of miRNAs with significant expression changes were determined. FIG. 5A illustrates expression changes of exemplary first miRNAs of an exemplary miRNA biomarker panel in healthy controls compared to CKD patients and MN, DN, IgAN, and FSGS/MCD subgroups, consistent with one or more exemplary embodiments of the present disclosure. The gene expression changes in the box plots were considered significant with probability p-value<0.05, p-value<0.01, and p-value<0.001.

Referring to FIG. 5A, expression of the miR-30a-5p (SEQ ID NO: 1) (FC=3.07) and the miR-486-5p (SEQ ID NO: 2) (FC=2.32) showed a significantly higher expressions in CKD patients compared to healthy individuals (p-value<0.01). The miR-29c-3p (SEQ ID NO: 3) (FC=0.52) and miR-200c-3p (FC=0.11) (SEQ ID NO: 4) also showed a significantly lower expression in the CKD patients compared to healthy individuals (p-value<0.05). Also, the miR-216b-5p (SEQ ID NO: 5) showed a significantly lower expression (FC=0.216) among all CKD patients compared to healthy ones (p-value<0.01). Therefore, validated biomarker miRNAs and their status for diagnosing CKD are miR-30a-5p↑, miR-486-5p↑, miR-29c-3p↓, miR-200c-3p↓, and miR-216b-5↓.

FIG. 5B illustrates expression changes of exemplary second miRNAs of an exemplary miRNA biomarker panel in healthy controls compared to all CKD patients and the MN, DN, IgAN, and FSGS/MCD subgroups, consistent with one or more exemplary embodiments of the present disclosure. The gene expression changes in the box plots were considered significant with probability p-value<0.05, p-value<0.01, and p-value<0.001.

In an exemplary embodiment, the higher expression level than the respective reference level may include an expression level higher than the respective reference level with a fold change of at least about 1.5 at a significance threshold of p<0.05. In an exemplary embodiment, the lower expression level than the respective reference level may include an expression level lower than the respective reference level with a fold change of up to about 0.55 at a significance threshold of p<0.05. Referring to FIG. 5B, in the DN subgroup, miR-126-3p (SEQ ID NO: 6) and miR-26a-5p (SEQ ID NO: 7) showed a significantly higher expression (FC=1.69) and a significant lower expression (FC=0.27) in the CKD patients compared to healthy controls (p-value<0.05). Also, miR-135b-5p (SEQ ID NO: 8) had a significantly lower expression (FC=0.27) in the CKD patients compared to the healthy individuals (p-value<0.05). Therefore, validated biomarker miRNAs and their status for diagnosing DN are miR-126-3p↑, miR-26a-5p↓, and miR-135b-5p↓.

In the IgAN subgroup, let-7b-5p (SEQ ID NO: 9) showed a significantly higher expression level (FC=8.2) compared to the healthy control (p-value<0.05). However, miR-26a-5p (SEQ ID NO: 7) did not show significant expression changes and had an unchanged expression level, which means that miR-26a-5p had an expression level with a fold change between 0.55 and 1.5 at a significance threshold of p<0.05 relative to the respective reference level. On the other hand, miR-135b-5p (SEQ ID NO: 8) also had a significantly lower expression level (FC=0.04) in comparison to the control (p-value<0.01). Therefore, validated biomarker miRNAs and their status for diagnosis of IgAN are let-7b-5p↑, miR-135b-5p↓.

In the FSGS/MCD subgroup, miR-135b-5p (SEQ ID NO: 8) showed a significantly higher expression level (FC=2.43) compared to healthy controls (p-value<0.05). On the other hand, miR-126-3p (SEQ ID NO: 6) also had a significantly lower expression level (FC=0.02) than the healthy controls (p-values<0.05). Therefore, validated biomarker miRNAs and their status for diagnosing the FSGS/MCD are miR-135b-5p↑ and miR-126-3p↓. In the MN subgroup, miR-126-3p (SEQ ID NO: 6) (p-value<0.05, FC=2.23) and let-7b-5p (SEQ ID NO: 9) (p-value<0.05, FC=2.96) showed higher expression level compared to healthy controls and. Therefore, validated biomarker miRNAs and their status for diagnosing MN are let-7b-5p↑ and miR-126-3p↓.

### Example 3: Diagnostic Sensitivity and Specificity of Exemplary MIRNA Biomarker Panel

In this example, diagnostic sensitivity and specificity of exemplary miRNA biomarker panel were evaluated using receiver operating characteristic (ROC) curves. The ROC curves were drawn by plotting a real positive rate (sensitivity) against a real negative rate (1−specificity) based on a cut-off point. The area under the ROC curve (AUC) estimates how well a particular miRNA may differentiate between the two groups (healthy and patient).

Here, sensitivity means the probability that a test's result is positive in patients and determines the diagnostic test's cut-off. The specificity is the probability that the test result is negative in healthy people. Both of these parameters are expressed as percentages and determine the diagnostic power of a test. Therefore, since the specificity compensates for the lower sensitivity in the ROC curve, AUC above 50% is acceptable in numerical diagnostic tests and shows that the test has the power to diagnose healthy people from patients. In this study, the ROC curve in SPSS was plotted for each of the miRNAs that showed significant dysregulation.

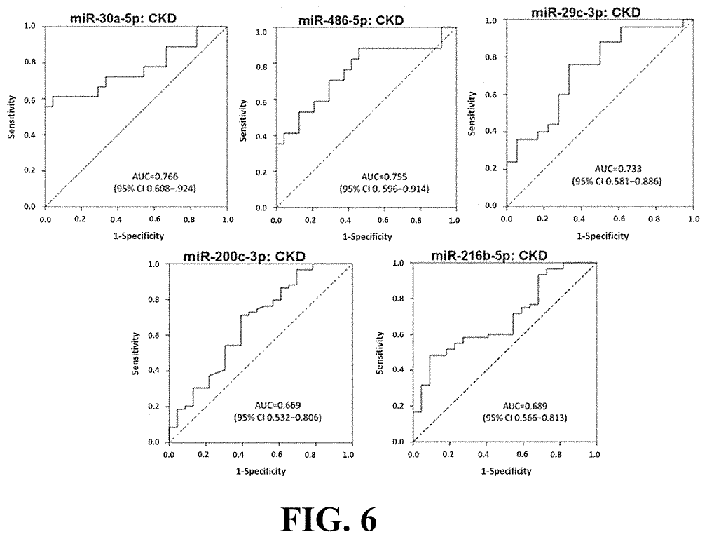

FIG. 6 illustrates receiver operating characteristic (ROC) curves of exemplary first miRNAs of an exemplary miRNA biomarker panel in CKD patients and healthy controls, consistent with one or more exemplary embodiments of the present disclosure. Referring to FIG. 6, the AUC of the miR-30a-5p (SEQ ID NO: 1) is higher than the others and has the highest diagnostic accuracy.

Also, the specificity and the sensitivity of the first miRNAs are as follows. The miR-30a-5p (SEQ ID NO: 1) has a sensitivity of about 0.611 and a specificity of about 0.958. The miR-486-5p (SEQ ID NO: 2) has a sensitivity of about 0.882 and a specificity of about 0.542. The miR-29c-3p (SEQ ID NO: 3) has a sensitivity of about 0.760 and a specificity of about 0.666. The miR-200c-3p (SEQ ID NO: 4) has a sensitivity of about 0.712 and a specificity of about 0.608. The miR-216b-5p (SEQ ID NO: 5) has a sensitivity of about 0.517 and a specificity of about 0.818. As a result, the miR-30a-5p (SEQ ID NO: 1) has the highest diagnostic specificity, and the miR-486-5p (SEQ ID NO: 2) has the highest diagnostic sensitivity.

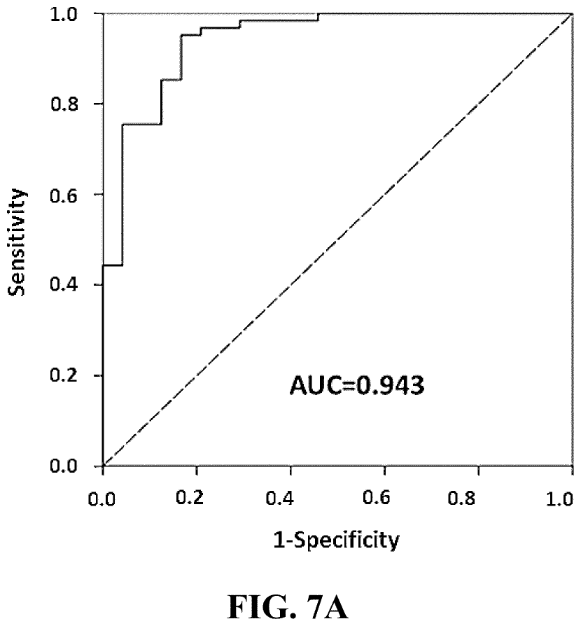

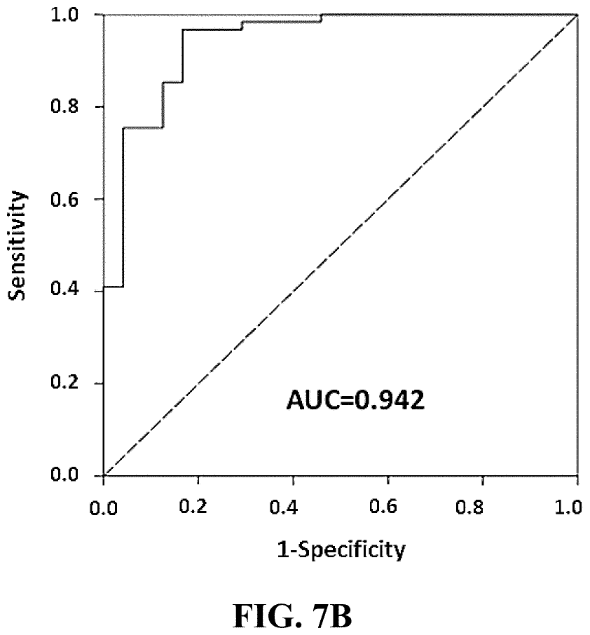

FIG. 7A illustrates combined ROC curves of all miRNAs of first miRNAs, including miR-30a-5p (SEQ ID NO: 1), miR-486-5p (SEQ ID NO: 2), miR-29c-3p (SEQ ID NO: 3), miR-200c-3p (SEQ ID NO: 4), and miR-216b-5p (SEQ ID NO: 5), consistent with one or more exemplary embodiments of the present disclosure. Referring to FIG. 7A, ROC results were combined in all five of the first miRNAs using logistic regression. The results show the accuracy of the prediction with AUC=0.943, which means combined use of all of the first miRNAs increases the CKD diagnosis's accuracy to 94%.

FIG. 7B illustrates combined ROC curves of four miRNAs of first miRNAs, including miR-30a-5p (SEQ ID NO: 1), miR-486-5p (SEQ ID NO: 2), miR-29c-3p (SEQ ID NO: 3), and miR-200c-3p (SEQ ID NO: 4), consistent with one or more exemplary embodiments of the present disclosure. Referring to FIG. 7B, the ROC results with conditional logistic regression and the estimated probability values, showed that use of four miRNAs of first miRNAs, including miR-30a-5p (SEQ ID NO: 1), miR-486-5p (SEQ ID NO: 2), miR-29c-3p (SEQ ID NO: 3), and miR-200c-3p (SEQ ID NO: 4) had an accuracy of prediction with AUC=0.942. Referring again to FIG. 7B, by measuring these four miRNAs of the first miRNAs without using miR-216b-5p (SEQ ID NO: 5), a similar diagnostic accuracy is obtained compared to FIG. 7A, in addition to reducing the cost of diagnosis.

Similarly, the highest AUC in the DN disease subgroup belongs to miR-29c-3p (SEQ ID NO: 3) (AUC=0.900 95% CI 0.767-1.00), in the IgAN disease subgroup belongs to miR-135b-5p (SEQ ID NO: 8) (AUC=0.869 95% CI 0.724-1.00), in the FSGS/MCD disease subgroup belongs to miR-200c-3p (SEQ ID NO: 4) (AUC=0.711 95% CI 0.554-0.868) and in the MN disease subgroup belongs to miR-486-5p (SEQ ID NO: 2) (AUC=0.824 95% CI 0.641-1.00).

With the help of logistic regression, ROC results of all miRNAs of exemplary miRNA biomarker panel in each of the CKD subgroups, including DN, IgAN, FSGS/MCD, and MN, were combined, and results confirmed the prediction accuracy with AUC of 0.958, 1, 0.846, and 0.943, respectively for each subgroup. The combined use of all miRNAs of exemplary miRNA biomarker panel in each subgroup of the disease significantly increases the diagnosis accuracy.

### Example 4: Examination of the Disease Progress Using Exemplary MIRNA Biomarker Panel

CKD is a chronic, progressive disease that can progress to further renal failure and ESRD (end-stage renal disease). Therefore, a biomarker that can statistically justify the disease's course can help disease management, evaluate responses to treatment, and evaluate the effect of drugs that are utilized. It can also be very effective in identifying biological pathways associated with the disease. In this example, the expression of exemplary miRNA biomarker panel during the CKD progression was examined.

Spearman correlation is a criterion for estimating the linear relationship between two variables and is determined by the coefficient ρ. Accordingly, in all samples for each miRNA, the significance of the relationships between interstitial fibrosis and tubular atrophy (IFTA), sclerosis, serum creatinine, proteinuria, glomerular filtration rate (GFR), and miRNA expression was determined based on the Spearman correlation. TABLE 1 presents the correlation between the miRNA expressions of exemplary miRNA biomarker panel, the histopathological parameters, and renal function indices in all CKD patients.

TABLE 1
Correlation between miRNA expression of exemplary miRNA biomarker
panel, histopathological parameters, and renal function indices
in all CKD patients. Significant correlations are highlighted.

| | | miR- | miR- | miR- | miR- | miR- | miR- | miR- | miR- | Let- |
|---|---|---|---|---|---|---|---|---|---|---|
| Marker | | 30a | 486 | 29c | 200c | 216b | 135b | 126 | 26a | 7b |
| IFTA | Correlation | .259 | .318* | −.334* | −.260* | −.234* | −.082 | −.069 | −.158 | .124 |
| | P-value | .082 | .029 | .023 | .019 | .034 | .479 | .545 | .155 | .273 |
| Sclerosis | Correlation | .219 | .246 | −.475** | −.303** | −.139 | −.164 | −.051 | .029 | .037 |
| | P-value | .143 | .096 | .001 | .006 | .212 | .155 | .657 | .795 | .744 |
| Serum | Correlation | .292* | .328* | −.452** | −.287** | −.328** | −.163 | −.118 | −.231* | .128 |
| creatinine | P-value | .049 | .025 | .002 | .009 | .003 | .157 | .300 | .036 | .259 |
| Proteinuria | Correlation | .267 | .137 | −.356* | −.160 | −.228* | .118 | −.113 | −.311** | .142 |
| | P-value | .073 | .359 | .015 | .155 | .039 | .309 | .319 | .004 | .208 |
| GFR | Correlation | −.377** | −.415** | .415** | .266* | .278* | .209 | .075 | .245* | −.087 |
| | P-value | .010 | .004 | .004 | .017 | .011 | .068 | .514 | .026 | .445 |

According to TABLE 1, there was a significant positive correlation between IFTA index and expression of miR-486-5p (SEQ ID NO: 2) and a significant negative correlation between IFTA index and miR-29c-3p (SEQ ID NO: 3), miR-200c-3p (SEQ ID NO: 4), and miR-216b-5p (SEQ ID NO: 5) expressions in all CKD patients. There was a significant negative correlation between sclerosis index and miR-29c-3p (SEQ ID NO: 3) and miR-200c-3p (SEQ ID NO: 4) expressions in all CKD patients. There was a significant positive correlation between serum creatinine parameter and miR-30a-5p (SEQ ID NO: 1) expression and miR-486-5p (SEQ ID NO: 2) expressions and a significant negative correlation between serum creatinine parameter and miR-29c-3p (SEQ ID NO: 3), miR-200c-3p (SEQ ID NO: 4), miR-216b-5p (SEQ ID NO: 5) and miR-26a-5p (SEQ ID NO: 7) expressions in all CKD patients.

Moreover, there was a significant negative correlation between proteinuria index and expression of miR-29c-3p (SEQ ID NO: 3), miR-216b-5p (SEQ ID NO: 5) and miR-26a-5p (SEQ ID NO: 7) in all CKD patients. There was a significant negative correlation between GFR index and expressions of miR-30a-5p (SEQ ID NO: 1), miR-486-5p (SEQ ID NO: 2) and a significant positive correlation between miR-29c-3p (SEQ ID NO: 3), miR-200c-3p (SEQ ID NO: 4), miR-216b-5p (SEQ ID NO: 5) and miR-26a-5p (SEQ ID NO: 7) expressions in all CKD patients.

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such away. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second, and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure and is not to be interpreted as reflecting an intention that the claimed implementations require more features than are expressly recited in each claim. Rather, as the following claims reflect, the inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in the light of the attached claims and their equivalents. Also, various modifications and changes may be made within the scope of the attached claims.

What is claimed is:

1. A method for diagnosing chronic kidney disease (CKD), the method comprising:
measuring an expression level of each microRNA (miRNA) of first miRNAs in a biological sample of a subject, the first miRNAs comprising miR-30a-5p (SEQ ID NO: 1), miR-486-5p (SEQ ID NO: 2), miR-29c-3p (SEQ ID NO: 3), and miR-200c-3p (SEQ ID NO: 4);
comparing the expression level of each miRNA of the first miRNAs with a respective reference level corresponding to each miRNA of the first miRNAs; and
determining CKD responsive to higher expression levels of the miR-30a-5p (SEQ ID NO: 1) and the miR-486-5p (SEQ ID NO: 2) and lower expression levels of miR-29c-3p (SEQ ID NO: 3) and miR-200c-3p (SEQ ID NO: 4) than the respective reference level.

2. The method of claim 1 further comprising comparing an expression level of miR-216b-5p (SEQ ID NO: 5) with the respective reference level,
wherein the miR-216b-5p (SEQ ID NO: 5) has a lower expression than the respective reference level in CKD.

3. The method of claim 1 further comprising determining a subgroup of the CKD, comprising:
measuring an expression level of each miRNA of second miRNAs in the biological sample of the subject, the second miRNAs comprising miR-126-3p (SEQ ID NO: 6), miR-26a-5p (SEQ ID NO: 7), miR-135b-5p (SEQ ID NO: 8), and let-7b-5p (SEQ ID NO: 9);
comparing the expression level of each miRNA of the second miRNAs with a respective reference level corresponding to each miRNA of the second miRNAs; and
determining the subgroup of CKD responsive to the status of each miRNA of the second miRNAs, the subgroup of CKD comprising one of diabetic nephropathy (DN), IgA nephropathy (IgAN), membranous nephropathy (MN), and focal segmental glomerulus nephropathy and minimal change disease (FSGS/MCD).

4. The method of claim 2, wherein determining diabetic nephropathy (DN) responsive to a higher expression level of the miR-126-3p (SEQ ID NO: 6) and lower expression levels of the miR-26a-5p (SEQ ID NO: 7) and the miR-135b-5p (SEQ ID NO: 8) than the respective reference level.

5. The method of claim 2, wherein determining IgA nephropathy (IgAN) responsive to a higher expression level of the let-7b-5p (SEQ ID NO: 9) and a lower expression level of the miR-135b-5p (SEQ ID NO: 8) than the respective reference level.

6. The method of claim 2, wherein determining membranous nephropathy (MN) responsive to higher expression levels of the let-7b-5p (SEQ ID NO: 9) and the miR-126-3p (SEQ ID NO: 6) than the respective reference level.

7. The method of claim 2, wherein determining minimal change disease and primary focal segmental glomerulosclerosis (MCD/FSGS) responsive to a higher expression level of the miR-135b-5p (SEQ ID NO: 8) and a lower expression level of the miR-126-3p (SEQ ID NO: 6) than the respective reference level.

8. The method of claim 1, wherein the higher expression level comprises an expression level higher than the respective reference level with a fold change of at least 1.5 at a significance threshold of p<0.05.

9. The method of claim 1, wherein the lower expression level comprises an expression level lower than the respective reference level with a fold change of up to 0.55 at a significance threshold of p<0.05.

10. The method of claim 1, wherein measuring the expression level of first miRNAs in the biological sample of the subject comprises measuring the expression level of first miRNAs in the biological sample by conducting at least one of an amplification-based method, a hybridization-based method, and a sequencing method.

11. The method of claim 9, wherein conducting at least one of an amplification-based method, a hybridization-based method, and a sequencing method comprises conducting at least one of real-time polymerase chain reaction (RT-PCR), an isothermal amplification method, a microarray assay, and next-generation sequencing (NGS).

12. The method of claim 1, wherein measuring the expression level of first miRNAs in the biological sample of the subject comprises measuring the expression level of first miRNAs in at least one of a urine sample, a blood sample, a serum sample, a plasma sample, and a kidney biopsy.

13. A kit for diagnosing chronic kidney disease (CKD), the kit comprising:
reagents for measuring an expression level of each microRNA (miRNA) of first miRNAs in a biological sample of a subject using a first set of oligonucleotides, the first set of oligonucleotides capable of specifically binding to the first miRNAs or their corresponding complementary DNA (cDNA), the first miRNAs comprising miR-30a-5p (SEQ ID NO: 1), miR-486-5p (SEQ ID NO: 2), miR-29c-3p (SEQ ID NO: 3), and miR-200c-3p (SEQ ID NO: 4)s.

14. The kit of claim 13, wherein the first miRNAs further comprise miR-216b-5p (SEQ ID NO: 5).

15. The kit of claim 13 further comprising:
reagents for measuring an expression level of each miRNA of second miRNAs using a second set of oligonucleotides, the second set of oligonucleotides capable of specifically binding to second miRNAs or their corresponding cDNA, the second miRNAs comprising: miR-126-3p (SEQ ID NO: 6), miR-26a-5p (SEQ ID NO: 7), miR-135b-5p (SEQ ID NO: 8), and let-7b-5p (SEQ ID NO: 9).

16. The kit of claim 15, wherein the reagents for measuring the expression level of each miRNA of the first miRNAs and the second miRNAs comprise reagents for measuring the expression level of each miRNA based on at least one of an amplification-based method, a hybridization-based method, and a sequencing method.

17. The kit of claim 16, wherein the reagents for measuring the expression level of each miRNA based on at least one of the amplification-based method, the hybridization-based method, and the sequencing method comprise reagents for measuring the expression level of each miRNA based on at least one of real-time polymerase chain reaction (RT-PCR), an isothermal amplification method, a microarray assay, and next-generation sequencing (NGS).

18. The kit of claim 15, wherein the first set of oligonucleotides and the second set of oligonucleotides comprise at least one of immobilized oligonucleotides and detectably labeled oligonucleotides.


SEQUENCE LISTING (10 sequences)

SEQ ID NO: 1 - RNA, 22 nt, Homo sapiens
uguaaacauccucgacuggaag

SEQ ID NO: 2 - RNA, 22 nt, Homo sapiens
uccuguacugagcugccccgag

SEQ ID NO: 3 - RNA, 22 nt, Homo sapiens
uagcaccauuugaaaucgguua

SEQ ID NO: 4 - RNA, 23 nt, Homo sapiens
uaauacugccggguaaugaugga

SEQ ID NO: 5 - RNA, 22 nt, Homo sapiens
aaaucucugcaggcaaauguga

SEQ ID NO: 6 - RNA, 22 nt, Homo sapiens
ucguaccgugaguaauaaugcg

SEQ ID NO: 7 - RNA, 22 nt, Homo sapiens
uucaaguaauccaggauaggcu

SEQ ID NO: 8 - RNA, 23 nt, Homo sapiens
uauggcuuuucauuccuauguga

SEQ ID NO: 9 - RNA, 22 nt, Homo sapiens
ugagguaguagguugugugguu

SEQ ID NO: 10 - RNA, 111 nt, Homo sapiens
gccgggagguugaacauccugcauagugcugccaggaaaucccuauuucauauaagagggggcuggcugguugcauauguaggaugucccaucucccagcccacuucguca